(19) United States
(12) Patent Application Publication
(10) Pub. No.: US 2022/0375578 A1
(43) Pub. Date: Nov. 24, 2022

(54) Healthcare Product Recall Management System

(71) Applicant: Oracle International Corporation, Redwood Shores, CA (US)

(72) Inventors: Manighandan VENKATRAMAN, Bangalore (IN); Soma Sekhar Srinivas SADARAM, Hyderabad (IN)

(21) Appl. No.: 17/382,724

(22) Filed: Jul. 22, 2021

(30) Foreign Application Priority Data
May 21, 2021 (IN) 202141022723

(51) Int. Cl. G16H 40/20; G06Q 30/00; G06Q 10/00; G06F 9/54; G06F 16/22

(52) U.S. Cl. CPC G16H 40/20; G06Q 30/014; G06Q 10/30; G06F 9/541; G06F 16/2282; G06Q 50/28

(57) ABSTRACT

Embodiments manage the recall of a healthcare product that includes a plurality of healthcare parts. Embodiments capture a recall notice for the healthcare product. Embodiments initiate by a locate engine a background process for locating each of the healthcare parts. Embodiments locate each of the healthcare parts using product traceability, where each the healthcare parts includes a traceability status of Periodic Automated Replenishment (“PAR”), in stock, expense, or inbound. Based on the product traceability, embodiments generate a sequence of tasks based on the traceability status, the tasks implementing a recall action. Embodiments monitor the completion of the tasks to verify that the recall notice can be closed.

### FIELD

One embodiment is directed generally to a computer system, and in particular to an integrated computer system that manages healthcare product recalls.

### BACKGROUND INFORMATION

A healthcare product recall is a request to locate and return, destroy or update, a healthcare product (e.g., a medical implant or pharmaceutical drug) after the discovery of safety issues or product defects that might endanger the consumer or put the maker/seller at risk of legal action.

Product recalls can occur at any time. It could be a low probability but high consequence situation. In addition, ever increasing and changing regulatory compliance applies added pressure to adopt an effective product recall management strategy and practices. Quick and effective tracking and communication of the product recall to all affected stakeholders within the supply chain is critical and challenging.

### SUMMARY

Embodiments manage the recall of a healthcare product that includes a plurality of healthcare parts. Embodiments capture a recall notice for the healthcare product. Embodiments initiate by a locate engine a background process for locating each of the healthcare parts. Embodiments locate each of the healthcare parts using product traceability, where each of the healthcare parts includes a traceability status of Periodic Automated Replenishment (“PAR”), in stock, expense, or inbound. Based on the product traceability, embodiments generate a sequence of tasks based on the traceability status, the tasks implementing a recall action. Embodiments monitor the completion of the tasks to verify that the recall notice can be closed.

### BRIEF DESCRIPTION OF THE DRAWINGS

FIG. 1 is a block diagram of overall functionality of a healthcare product recall system in accordance to embodiments of the invention.

FIG. 2 is a block diagram of one or more components of the system of FIG. 1 in the form of a computer server/system in accordance with an embodiment of the present invention.

FIG. 3 is an overall flow diagram of is a flow diagram of the functionality of the healthcare product recall module of FIG. 3 for performing healthcare product recall in accordance with one embodiment.

FIG. 4 is a block diagram illustrating the integration of the healthcare product recall system with 3rd parties in accordance to embodiments of the invention.

FIG. 5 is a block diagram illustrating the integration of the healthcare product recall system with source systems to capture recall notices in accordance to embodiments of the invention.

FIG. 6 is a block diagram illustrating the integration of the healthcare product recall system with source systems to capture recall notices in accordance to other embodiments of the invention.

FIG. 7 is a flow diagram illustrating the integration of the healthcare product recall system with RF-SMART to perform a healthcare product recall count in accordance to other embodiments of the invention.

FIG. 8 is a flow diagram illustrating the integration of the healthcare product recall system with 3rd party systems to trace recalls and take disposal actions in accordance to other embodiments of the invention.

FIG. 9 illustrates specialty data structures implemented in an embodiments of the invention.

FIG. 10 illustrates the contents of the recall notices in accordance with embodiments.

FIG. 11 is a flow diagram of the recall parts process in accordance to embodiments of the invention.

FIG. 12 illustrates functionality of the work area that provides a user with guided actions and appropriate actions to be taken on a recall notice based on its status and helps the user navigate to different user interfaces to manage the notices in accordance to embodiments.

FIG. 13 illustrates functionality of the locate recalled parts engine for locating recalled parts that are not tracked using a lot or serial number in accordance to embodiments.

FIG. 14 illustrates functionality of the locate recalled parts engine for locating recalled parts that do not have an internal item number in accordance to embodiments.

### DETAILED DESCRIPTION

Embodiments provide an integrated recall management system for the healthcare industry that uses novel interfaces to integrate third party applications and provides trace details for a specific item and/or location and trace details for tasks across all locations.

In addition to problems associated with product recalls in general, product recalls for healthcare products have differences that result in unique problems. For example, in healthcare, for healthcare products, the individual healthcare parts are typically not tracked using a lot number, serial number or internal item number within an inventory management system or enterprise resource planning (“ERP”) system. Instead, healthcare product parts are generally stocked in non-quantity tracked locations and therefore, merely using on-hand quantity tracking in an inventory management system will not help in tracing the recalled parts.

In the automotive industry, a product recall can be tracked by serial numbers using inventory on-hand balances as the parts would exist in quantity tracked locations and using install base records if the parts are already sold. In contrast, in the healthcare industry, parts generally exist in Periodic Automated Replenishment (“PAR”) locations, which are essentially non-quantity tracked locations or issued to expense locations which are the point of consumption in the respective departments without any serial or lot references within ERP that cannot be tracked using inventory on-hand balances. Therefore, embodiments include a “Locate Recalled Parts” engine that includes the capability of tracking the recalled parts lying in non-quantity tracked locations and parts that are not lot/serial controlled and not maintained with unique item number in an item master. Further, recalled parts that are in transit or available as a scheduled supply or lying-in receiving dock before getting shelved in the warehouse get tracked in embodiments and the respective stakeholders are alerted so that they do not receive those parts for consumption but instead would quarantine them.

A healthcare product recall is a request to quarantine and dispose a product after the discovery of any defects with the product, health hazards, or safety issues that might endanger the consumer or put the maker/seller at risk of legal action.

Managing the recall originating in the supply chain is a critical path for consumer safety. However, as discussed, there are multiple specialized needs in managing a product recall specific to healthcare products, including the following:

Automating receipt, handling, and processing of recall notices from multiple stakeholders: Untimely receipt of information about a product recall due to manual notification methods with multiple stakeholders involved in the supply chain such as manufacturer, distributor, regulatory authorities and third party solution providers who provide product alerts and recalls and internal stakeholders who consume those affected goods.

Technique for effective tracking and trace to ensure timely identification and locating affected goods: Limited visibility into what lots or serials were consumed or in which locations the affected goods could potentially exist. Delay in identification of affected goods increases the risk of consumption of those goods especially in the healthcare industry where the patient life is put at risk. Multiple execution systems are involved in the supply chain where product master, procurement of goods, inventory and order fulfilment is managed in ERP, drugs or devices issued to patients and products implanted on patients are tracked in the Point of Sale (“POS”) and the Electronic Health Record (“EHR”) respectively. Systems do not communicate with one another nor have a common value to allow tracing the recalls up to the end consumer

Monitoring disposition action: No visibility and control over what actions are taken on the recalled goods that are identified and hence no accountability on the disposition action to be carried out for the affected goods.

Known ERP systems require users to follow a large amount of complex manual processes in an attempt to address the above problems related to healthcare product recalls. Specifically, users of known ERP systems receive manual notification on recall from multiple sources and then need to inform the internal stakeholders manually. Users further need to verify whether the recalled lots or serials have been procured by the organization and identify the locations where these affected parts exist, which is a cumbersome and time-consuming activity. By the time all these locations are identified, there is a high possibility of the recalled parts getting consumed, putting the consumer at risk. Users need to then manually follow up with warehouse personnel to dispose the affected parts and keep track on the disposition details. If the affected product is not in stock, users then review POS and EHR or implant logs manually to identify whether any patient has adverse impact due to the recalled product and take corrective action.

In contrast to known solutions for implementing healthcare product recalls, including using existing functionality in an ERP system, such as an ERP system from Oracle Corp.®, embodiments provide the following novel functionality: (1) Capturing of product recalls from different sources that includes regulatory authority, manufacturers, distributors or any third-party solution providers; (2) Locating recalled parts based on on-hand inventory balances and historical purchases; (3) Assigning tasks and align resources to quarantine the affected goods; and (4) Monitoring disposal tasks to completion to ensure all impacted goods are disposed.

Reference will now be made in detail to the embodiments of the present disclosure, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, and circuits have not been described in detail so as not to unnecessarily obscure aspects of the embodiments. Wherever possible, like reference numbers will be used for like elements.

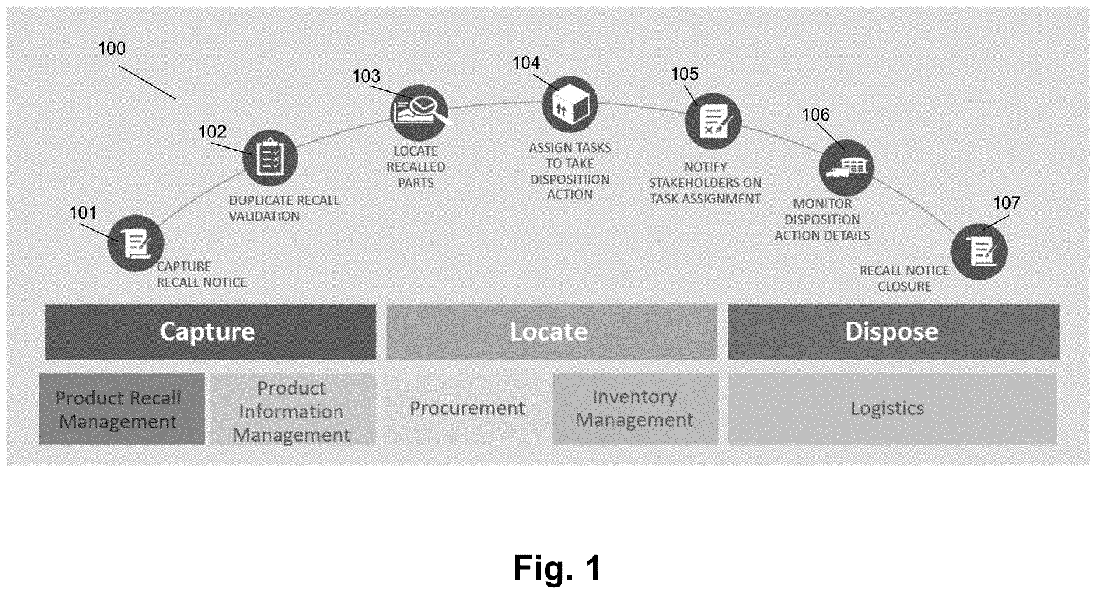

FIG. 1 is a block diagram of overall functionality of a healthcare product recall system 100 in accordance to embodiments of the invention. Embodiments manage recall notices by capturing the recall notice 101 and duplicate recall validation 102 (i.e., resolving duplicate recall notices). The recall notice is created through a user interface, a Representational State Transfer (“REST”) Application Programming Interface (“API”) or via a bulk import through File Based Data Import (“FBDI”). Embodiments can be integrated with manufacturers, distributors, regulatory authority bodies such as the Food and Drug Administration (“FDA”) or subscription service providers who send product alerts and recalls to enable getting up-to-the-minute product recalls.

In embodiments, a recall notice is always captured for a manufacturer part number which acts as a unique reference across different sources regardless of who initiates the recall, along with the lot and serial numbers that are recalled. Embodiments provide support for description-based items where the internal item number is not maintained in the product master.

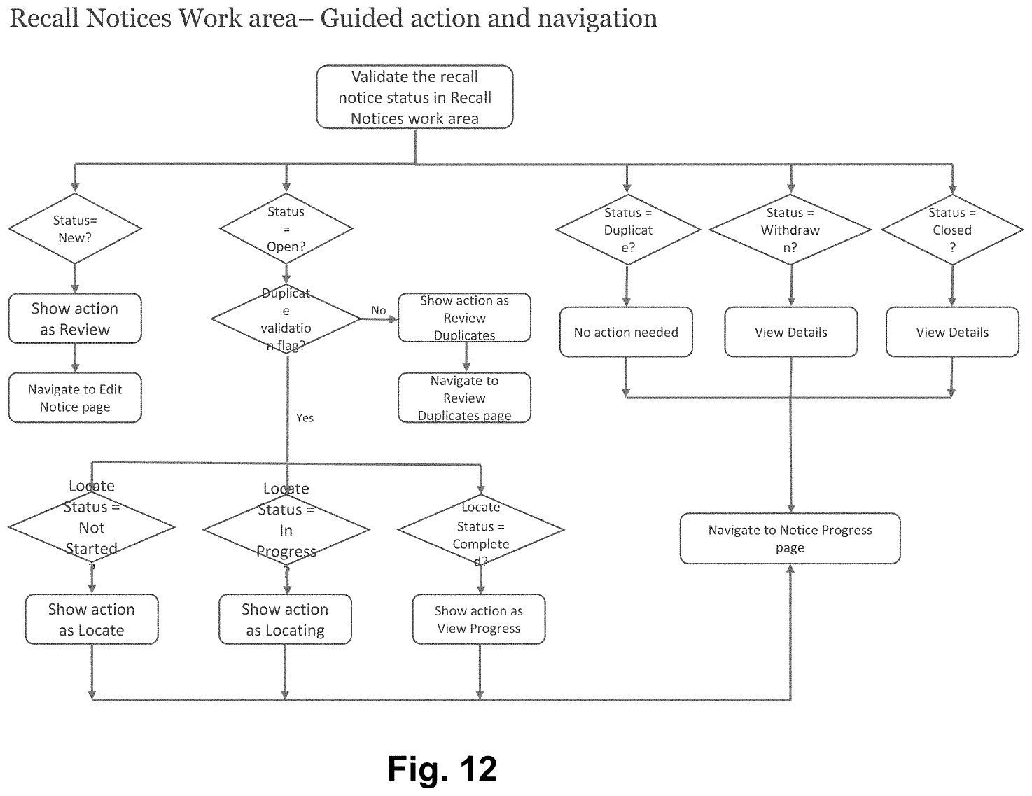

In embodiments, product recalls captured from multiple sources are centrally managed through a work area which guides the user with appropriate actions to be taken on a recall notice based on its status and helps the user navigate to different user interfaces to manage the notices (details disclosed in FIG. 12 below). Embodiments include searching so that the recall notices can be retrieved. The searching includes advanced capabilities such as keyword search, fuzzy matching, and multiple search criteria with Boolean operators. Embodiments avoid redundancy and reduce recall to disposition cycle time by resolving potential duplicates that can get created when recall notices are captured from multiple sources at 102.

Specifically, embodiments include a workbench with advanced search capabilities with fuzzy match, auto suggestions, recent searches and filters that provides visibility on the recall notices that automatically get imported and notices progressing with different statuses. Embodiments further guide a user with appropriate actions to be taken on a recall notice based on its status and helps in navigating to different user interfaces to manage the notices. For example, if a recall notice is imported very recently as a “New” status, embodiments will suggest the user to review the notice and publish it. However, if the notice is already published and having the status as Open, embodiments will provide a suggestion to proceed with the next step of resolving the duplicates.

Embodiments use an elastic search technique where the recall notice attributes are ingested into a search server, also referred to as “Global Search” from Oracle Corp. The Global Search, for Oracle Enterprise Applications, allow each application having search requirements to define their searchable business objects and enable full-text, real-time search capabilities on them. It provides design-time capabilities to define search indexes and provides run-time capabilities to push these index payloads into an Elasticsearch search engine and query them via REST and Java APIs.

When a search is performed on the user interface, recall notices are retrieved based on the keywords that match with the indexed attributes. Guided navigation is provided in this user interface such that user can take appropriate action by navigating to the respective page as per the status of the recall notice.

Embodiments further locate recalled parts 103 and assign tasks 104.

Embodiments locate the recalled parts in the supply chain based on on-hand inventory balances for parts that can be stocked in quantity-tracked locations and based on historical purchases for parts that are received in non-quantity tracked locations. Recalled parts are traced by lot or serial if the item is lot or serial controlled, or traced by date range when the parts are not lot/serial controlled.

Embodiments include a locate recalled parts engine that is implemented as a background process which does the track and trace of recalled parts in the entire supply chain with different traceability statuses. Recalled parts that are in transit or available as a scheduled supply or lying-in receiving dock before getting shelved in the warehouse are traced as inbound. Recalled parts that exists in quantity tracked locations are identified based on stock availability and shown as in stock. Examples of transaction history include various transaction types, including Transfer Order Issue, Account Alias Issue, Movement Request Issue and User Defined Miscellaneous Issue, used to issue the material from stock to expense locations. Recalled parts that are issued to PAR locations are identified based on transaction history and shown as PAR with an estimated quantity. Recalled parts that are issued to expense locations as well gets identified based on transaction history and traced as expense with an estimated quantity.

Once the recalled parts are located at 103, embodiments assign a corresponding pre-defined set of tasks in a sequence based on traceability status and the replacement type captured in the recall notice at 104. Tasks include—Count, Deliver, Disposal, Debit Memo and Instructions. Count task refers to performing a recall count in the identified locations and move the counted parts to a quarantine location. Deliver task is meant to deliver the recalled parts to quarantine location. Disposal task refers to performing a return to vendor or scrap disposal. Debit memo task is meant for creating a debit note against the disposal performed for the recalled parts. Instructions task is meant to carry out the recall instructions like device correction or labelling changes.

Count tasks can be grouped by department represented by inventory organization or location or sub-inventory as per the configuration. Tasks get progressed automatically (e.g., marked as closed when completed) based on disposition details captured while performing the count and disposal activities. Embodiments allow a user to review the trace details for a specific item by selecting its different traceability statuses. Embodiments further allow a user to review the trace details across the recall notice for a specific task. For example, a user can see in which departments the count task is in progress and in which departments the task is completed. A user can also review the trace details for a specific inventory organization or sub-inventory.

Embodiments allow a user to configure business rules to send the notifications to respective stakeholders to carry out the recall tasks. Whenever a task becomes in progress, a notification will be sent to the user at 105 as defined in the rules so that they can start performing the required actions.

Embodiments monitor disposition details at 106 and the closure of the recall notice at 107. Based on the notifications received, users can start performing the recall count in all identified locations that include quantity tracked, PAR and expense locations. The recall count quantity can be reported through interfaces including an RF-SMART mobile inventory solution that utilizes mobile barcoding or through the “Oracle Visual Builder Plug-in” for Excel. Recall count can also be reported to an inventory management system such as “Oracle Inventory Management” through any third-party application by leveraging the Inventory Count REST API.

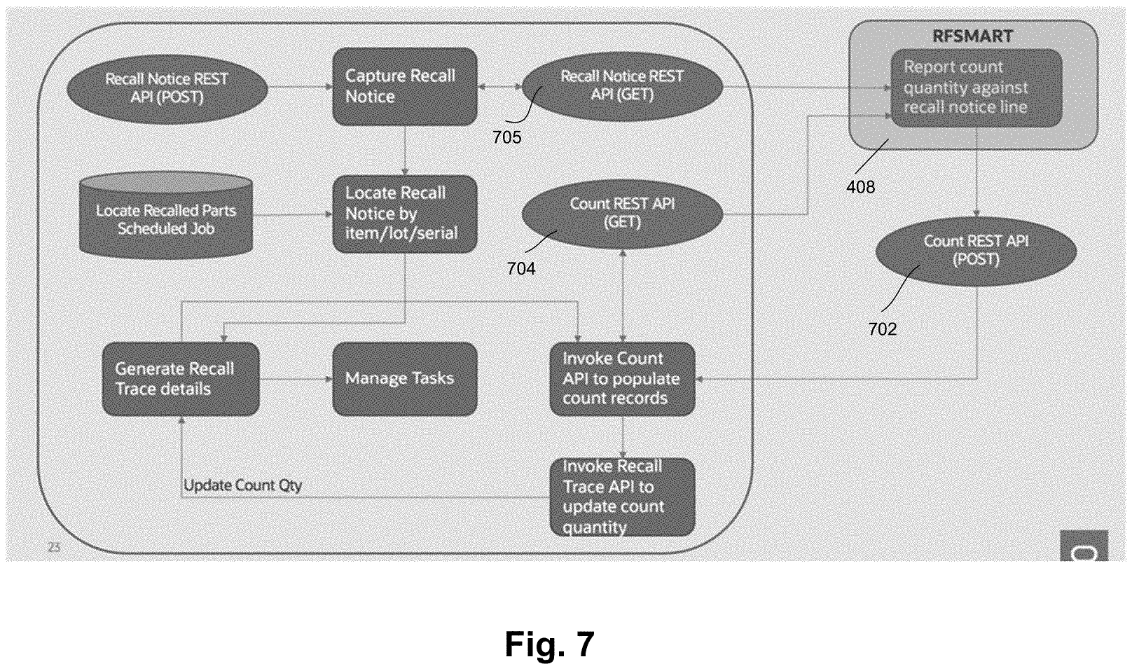

Reporting the count quantity automatically transfers the stock of counted parts to a quarantine location which is generally a non-reservable recall sub-inventory so that materials moved to this recall sub-inventory can no longer be consumed. Once the count quantity is reported in all identified sub-inventories or stock locators for all lots/serials, the corresponding task will get marked as completed automatically. Specifically, when the count quantity is reported, the count details get updated in the database against each identified stock-keeping unit (“SKU”) through a patch operation performed using the Inventory Count REST API. This in turn invokes the recall trace API to update the count quantity against the recall trace details stored in recall management schema based on which the count tasks move to closed status (shown below in FIG. 7).

Recalled parts that are counted and quarantined can be scrapped at the facility or returned to the supplier depending on the replacement type. Disposition details get updated automatically and disposition tasks gets marked as completed when all the counted quantities are returned. On completion of the disposal task, a debit memo task gets initiated and users are notified to create a debit memo for the return of recalled parts. Once notified, the debit memo task will be marked as completed.

When all the tasks assigned to a recall notice line get marked as completed, the recall notice line gets closed automatically. This ensures that a recall line is closed only after all affected goods are quarantined and disposed. When all the lines in a recall notice get closed, the recall notice is marked as closed. Embodiments can use the recall notice REST services to integrate the EHR or the POS with an ERP to track recalled parts that are already consumed by the patients and take necessary corrective action.

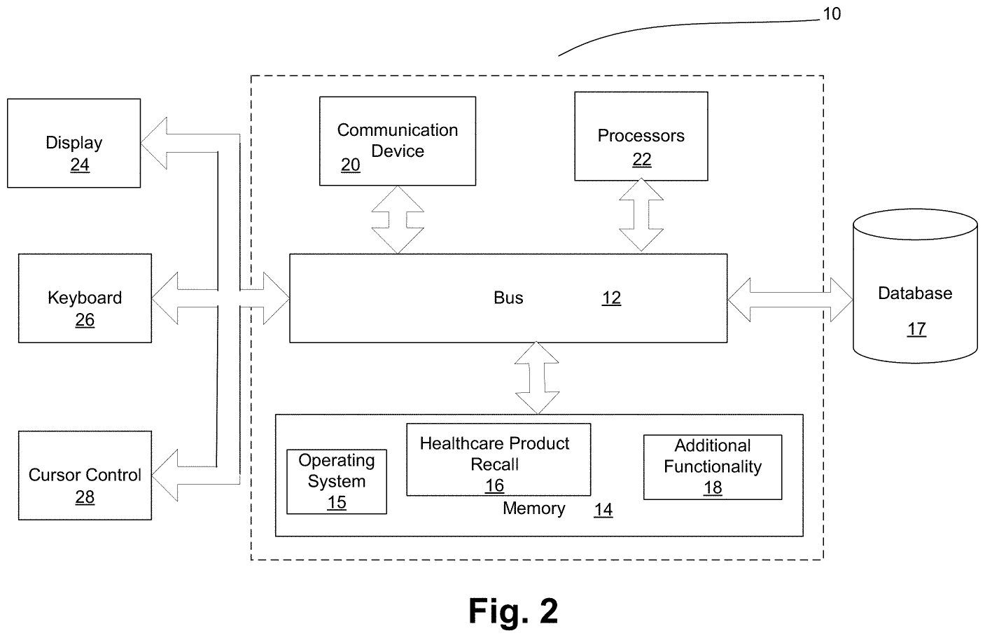

FIG. 2 is a block diagram of one or more components of system 100 of FIG. 1 in the form of a computer server/system 10 in accordance with an embodiment of the present invention. Although shown as a single system, the functionality of system 10 can be implemented as a distributed system. Further, the functionality disclosed herein can be implemented on separate servers or devices that may be coupled together over a network. Further, one or more components of system 10 may not be included. System 10 can be used to implement any of the components/elements/functionality shown in FIG. 1 and/or interact with any of the components.

System 10 includes a bus 12 or other communication mechanism for communicating information, and a processor 22 coupled to bus 12 for processing information. Processor 22 may be any type of general or specific purpose processor. System 10 further includes a memory 14 for storing information and instructions to be executed by processor 22. Memory 14 can be comprised of any combination of random access memory (“RAM”), read only memory (“ROM”), static storage such as a magnetic or optical disk, or any other type of computer readable media. System 10 further includes a communication device 20, such as a network interface card, to provide access to a network. Therefore, a user may interface with system 10 directly, or remotely through a network, or any other method.

Computer readable media may be any available media that can be accessed by processor 22 and includes both volatile and nonvolatile media, removable and non-removable media, and communication media. Communication media may include computer readable instructions, data structures, program modules, or other data in a modulated data signal such as a carrier wave or other transport mechanism, and includes any information delivery media.

Processor 22 is further coupled via bus 12 to a display 24, such as a Liquid Crystal Display (“LCD”) and includes a microphone for receiving user utterances. A keyboard 26 and a cursor control device 28, such as a computer mouse, are further coupled to bus 12 to enable a user to interface with system 10.

In one embodiment, memory 14 stores software modules that provide functionality when executed by processor 22. The modules include an operating system 15 that provides operating system functionality for system 10. The modules further include a healthcare product recall module 16 that implements healthcare product recall functionality, and all other functionality disclosed herein. System 10 can be part of a larger system. Therefore, system 10 can include one or more additional functional modules 18 to include the additional functionality. In one embodiment, module 18 implements an ERP, and module 16 is ether additional functionality integrated with the ERP, or interacts with the ERP functionality. In other embodiments, module 18 implements “Manufacturing and Supply Chain Materials Management” from Oracle Corp., and the functionality of module 16 is an added feature. A file storage device or database 17 is coupled to bus 12 to provide centralized storage for modules 16 and 18. In one embodiment, database 17 is a relational database management system (“RDBMS”) that can use Structured Query Language (“SQL”) to manage the stored data.

In one embodiment, particularly when there are a large number of distributed files at a single device, database 17 is implemented as an in-memory database (“IMDB”). An IMDB is a database management system that primarily relies on main memory for computer data storage. It is contrasted with database management systems that employ a disk storage mechanism. Main memory databases are faster than disk-optimized databases because disk access is slower than memory access, the internal optimization algorithms are simpler and execute fewer CPU instructions. Accessing data in memory eliminates seek time when querying the data, which provides faster and more predictable performance than disk.

In one embodiment, database 17, when implemented as an IMDB, is implemented based on a distributed data grid. A distributed data grid is a system in which a collection of computer servers work together in one or more clusters to manage information and related operations, such as computations, within a distributed or clustered environment. A distributed data grid can be used to manage application objects and data that are shared across the servers. A distributed data grid provides low response time, high throughput, predictable scalability, continuous availability, and information reliability. In particular examples, distributed data grids, such as, e.g., the “Oracle Coherence” data grid from Oracle Corp., store information in-memory to achieve higher performance, and employ redundancy in keeping copies of that information synchronized across multiple servers, thus ensuring resiliency of the system and continued availability of the data in the event of failure of a server.

In one embodiment, system 10 is a computing/data processing system including an application or collection of distributed applications for enterprise organizations, and may also implement logistics, manufacturing, and inventory management functionality. The applications and computing system 10 may be configured to operate with or be implemented as a cloud-based system, a software-as-a-service (“SaaS”) architecture, or other type of computing solution.

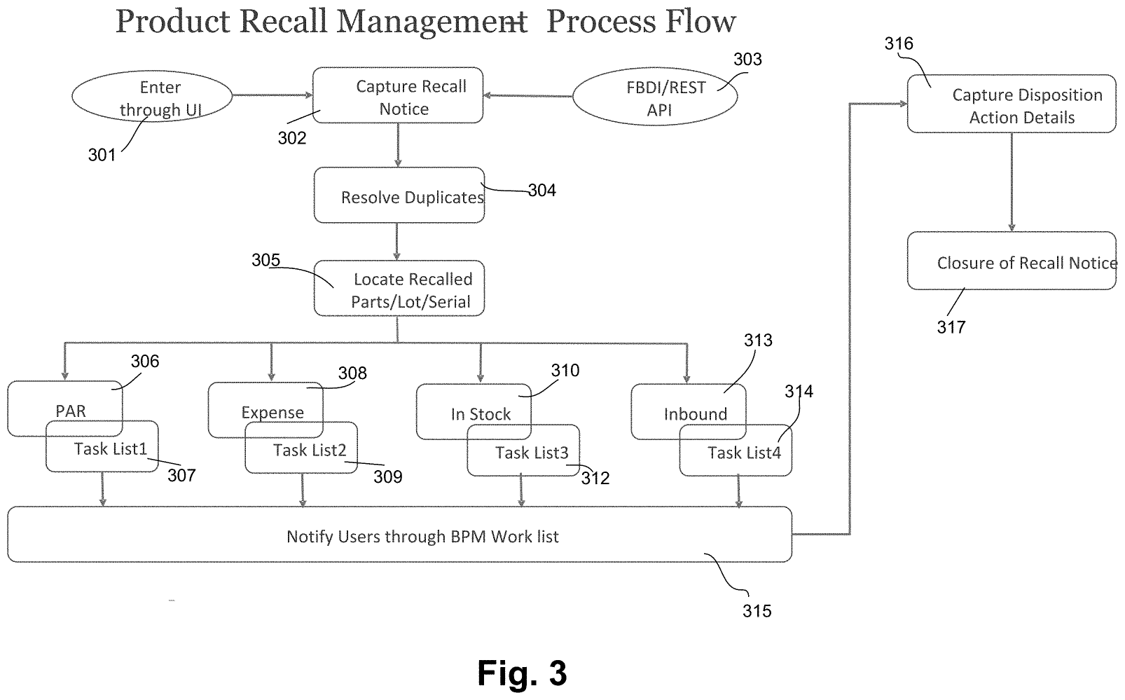

FIG. 3 is an overall flow diagram of the functionality of healthcare product recall module 16 of FIG. 3 for performing healthcare product recall in accordance with one embodiment. In one embodiment, the functionality of the flow diagram of FIG. 3 (and FIGS. 3, 7, 11 and 12 below) is implemented by software stored in memory or other computer readable or tangible medium, and executed by a processor. In other embodiments, the functionality may be performed by hardware (e.g., through the use of an application specific integrated circuit (“ASIC”), a programmable gate array (“PGA”), a field programmable gate array (“FPGA”), etc.), or any combination of hardware and software.

At 302, the recall notice is captured either through entering it in “Create Recall Notice” UI 301, File Based Data Import (“FBDI”) 303 as a mass upload through a macro enabled excel, or REST API 303. The recall notice includes a header and line section where the header includes a recall notice number, the source from where the recall notice was received, the initiation date, the closing date, manufacturer details, the recall notice type, classification based on severity of the impact that the recalled product has on consumers, potential risks and recall instructions and replacement type. The line section includes manufacturer part details, internal item details, lot and serial details.

There is a possibility that different sources can issue a recall notice for the same product. For example, a manufacturer of a particular product issues a recall notice once the manufacturer realizes that there is a product defect. A regulatory authority such as the FDA also can issue a recall notice for the same product based on complaints reported by consumers. In such a case, at 304 potential duplicate recall notices will be automatically suggested based on certain criteria, such as common product names, identifier numbers, manufacturer name, trading partner party number, source document reference, etc., after the recall notice is published. These potential duplicates will be marked as duplicate and no further action will be taken on those lines.

At 305, to identify the recalled parts in the supply chain, a “Locate” action will be initiated on the recall notices. This launches a scheduled job in the background which locates the recalled parts of the healthcare product with different product traceability statuses shown below depending on where those parts exist in the supply chain.
PAR (306): Parts issued to non quantity tracked sub-inventories;
IN STOCK (310): Parts lying in quantity tracked sub-inventories;
EXPENSE (308): Parts received in expense locations (departments) for consumption;
INBOUND (313): Parts lying in the receiving dock and in transit;
SOLD (not shown): Parts that are already sold to Customers against Sales Order.

Product traceability details are displayed in the form of header and child structure when a drill down is done on the recall notice lines. Traceability status is stored in a database. In embodiments, depending on the transaction types used and the recalled parts that are transacted and the locations where the recalled parts could potentially reside, traceability statuses exists as pre-defined lookup values. The header section includes product traceability status, warehouse, location, sub-inventory (optionally based on configuration) and total transaction quantity. The child section includes sub-inventory, stock locator and transaction details such as the source transaction type, source transaction number, on hand quantity/transaction quantity against each transaction.

Once the traceability status is identified and corresponding source transaction details are retrieved (i.e., the transaction history), a sequence of tasks will get automatically assigned to the recall notice lines (307, 309, 312, 314). This task assignment is primarily determined based on the product traceability status and the replacement type. The recalled parts are identified with different traceability statuses that have been defined as standard lookup values based on the transaction history and the locations where those parts reside.

Once the tasks get assigned, at 315 notifications are routed to appropriate stakeholders within the enterprise to perform necessary actions based on the task initiated. A business process management (“BPM”) work list is used to route the notifications where users can configure the rules to route the notification to users or roles based on different attributes pertaining to recall management as per their business requirement.

As instructed in the notification, users can perform the relevant action which results in certain transactions such as physical count reporting, moving the material to quarantine location, return to vendor or perform a scrap and updates the task status as “Completed”. Transaction details are also captured and shown as a disposition summary at 316. Once the first task in the sequence gets closed, the subsequent tasks get initiated and trigger notifications. Once all the tasks get completed, the recall notice line is closed at 317 and finally the status at the recall notice header comes to a closure.

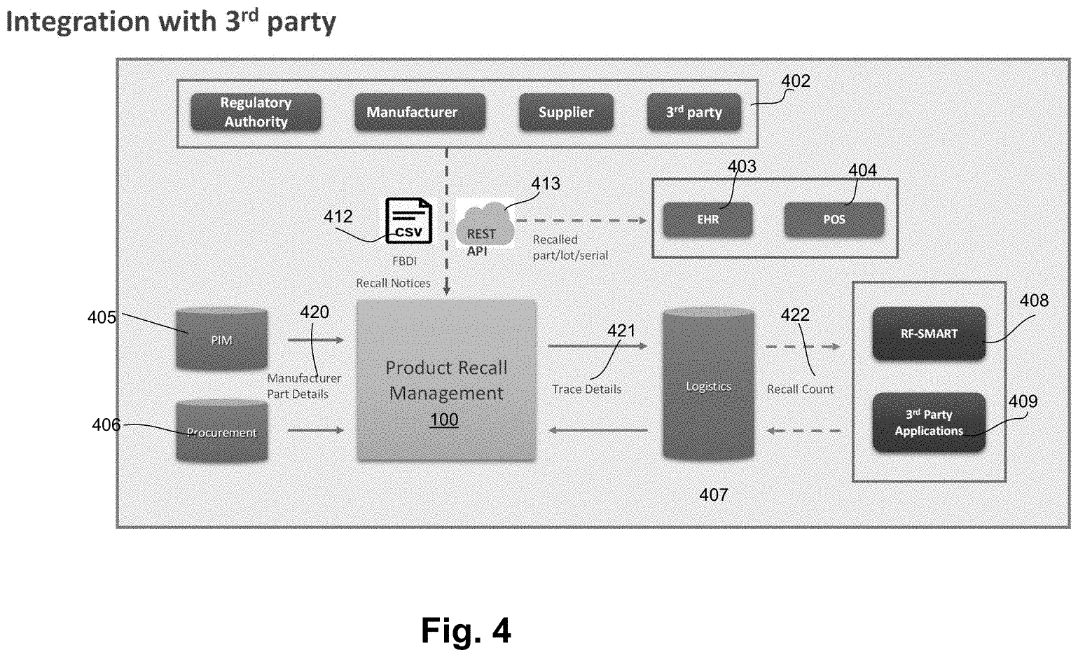

FIG. 4 is a block diagram illustrating the integration of healthcare product recall system 100 with 3^{rd} parties in accordance to embodiments of the invention. In embodiments, healthcare product recall system 100 (shown as product recall management 100) seamlessly integrates with other Supply Chain Applications from Oracle Corp. to implement a Recall to Disposition process and also integrates with partner applications and third-party applications to enable the capture of recall notices from different sources and capturing the disposition of recalled parts.

In embodiments, integration is implemented, at least in part, using “Service Oriented Architecture Cloud Service” (“SOA CS”) or Oracle Integration Cloud Service (“OIC”) from Oracle Corp. to automatically capture the recall notices 402 issued by regulatory authorities, manufacturers, suppliers or subscription services such as “RASMAS” into product recall management 100 using either file-based data import (“FBDI”) 412 or a Recall Notices REST API 413.

Product recall management 100 interacts with ERP modules. In connection with ERP from Oracle Corp., Oracle Product Information Management Cloud (“PIM”) 405 is used to get the inventory items and trading partner items for manufacturer part numbers. Oracle Procurement Cloud 406 is used to get the manufacturer part numbers from the blanket agreements. Once the recall parts are located at 420, the trace details 421 are sent to Oracle Inventory 407. The details of counting and disposal of the recalled parts are received from Oracle Inventory and Oracle Receiving in embodiments.

Product recall management 100 integrates with the RF-SMART 408 remote reader in embodiments, which enables recall count 422 and transfer of material to quarantine locations based on barcode scanning with a handheld device. This integration is implemented using Inventory Count Task REST services that supports interfacing the traceability details to RF-SMART and capturing the recall count details back to close the recall tasks after ensuring all the recalled parts are quarantined. These REST services in other embodiments can be used to perform recall count and disposition using any third-party warehouse execution systems 409 with an extension on the client side of warehouse management applications.

In embodiments, SOA CS or OIC is used to identify the recalled parts that are tracked in external applications such as EHR 403 and POS 404 once issued out of inventory in ERP, and take corrective actions required for disposal using Recall part/lot/serial REST services 413.

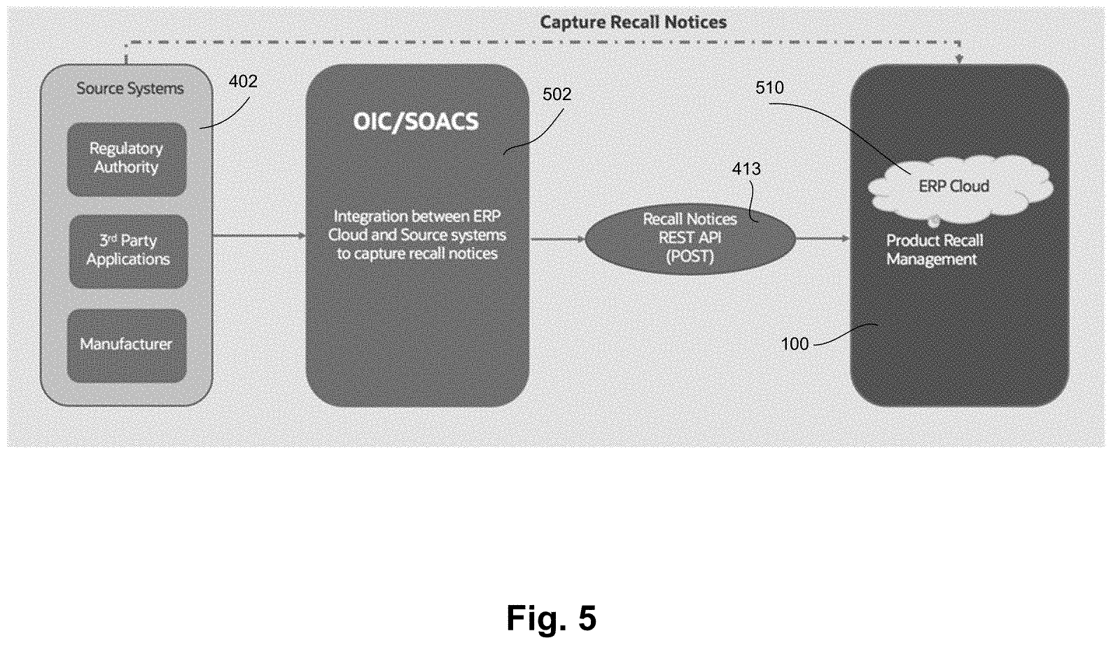

FIG. 5 is a block diagram illustrating the integration of healthcare product recall system 100 with source systems to capture recall notices in accordance to embodiments of the invention. Embodiments provide Recall Notice REST API services 413 that can be leveraged by the external parties 402 such as regulatory authorities and manufacturers who initiates the recall to import the recall notices. Embodiments implement a middleware platform 502 having SOA CS or OIC which allows a subscription to be done to the business events provided by the external applications 402 or their API that can be invoked through a scheduled orchestration as a batch process to get the recall notice data automatically whenever a recall notice is issued. This source data from external applications is mapped to the target, which is the payload attributes required by the recall notice REST API 413 within the middleware platform and this REST API can be invoked to create the recall notices in conjunction with Oracle ERP Cloud 510 in one embodiment.

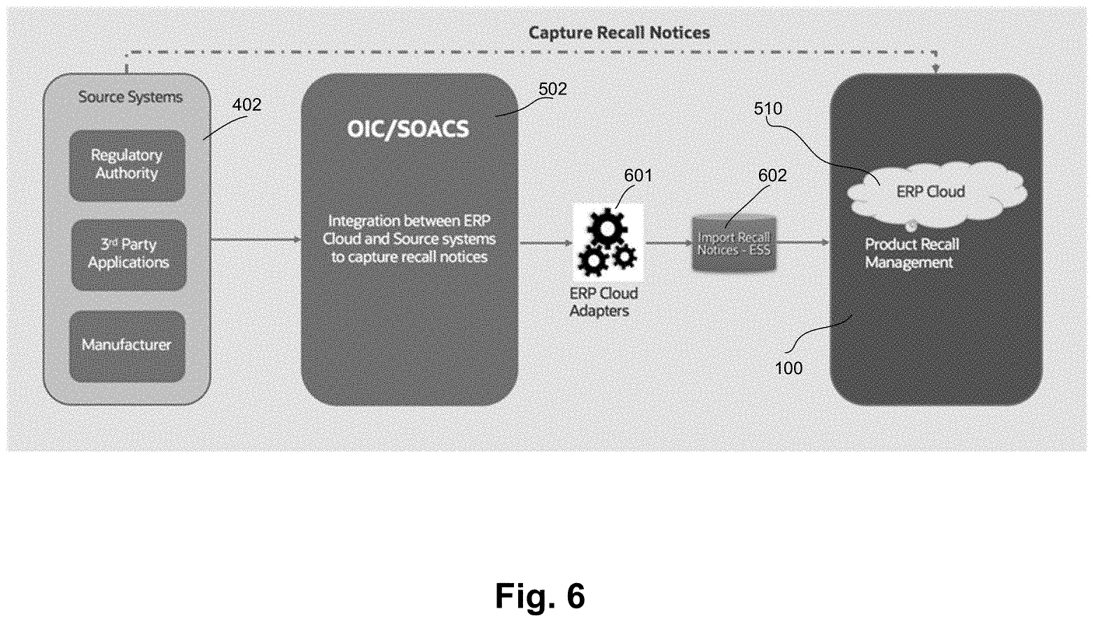

FIG. 6 is a block diagram illustrating the integration of healthcare product recall system 100 with source systems to capture recall notices in accordance to other embodiments of the invention. In embodiments, a file based data import (“FBDI”) process is used to implement bulk import of recall notices from external parties 402 such as regulatory authorities and manufacturers who initiates the recall. Embodiments implement a middleware platform 502 having SOA CS or OIC which allows a subscription to be done to the business events provided by the external applications 402 or their API that can be invoked through a scheduled orchestration as a batch process to get the recall notice data automatically whenever a recall notice is issued. This source data from external applications 402 is mapped to the target which are the attribute columns as prescribed in the FBDI template within the middleware platform to prepare a.csv file. With the help of ERP Cloud adapters 601, the csv file is placed in a Universal Content Manager (“UCM”) 602 and gets imported automatically into system 100 and ERP Cloud 510. UCM is a common repository where the files populated through external sources can be stored so that the scheduled processes (i.e., via Oracle Enterprise Scheduler or any other scheduler) in ERP can pick the file and import them into the ERP cloud.

FIG. 7 is a flow diagram illustrating the integration of healthcare product recall system 100 with RF-SMART to perform a healthcare product recall count in accordance to other embodiments of the invention. The recalled parts traced by the Locate Recalled Parts engine 103 of embodiments can be an estimated quantity that could possibly exist in identified locations given the constraints of non-quantity tracked locations, and parts not tracked with item number, lot or serial. The feature also offers a physical count mechanism where the warehouse personnel can report the count quantity against each identified location for each affected part. When the warehouse person is alerted on the recall, he/she can just go to the respective location and make an inquiry using the RF-SMART system 408, which is a handheld device that supports bar code scanning and performing inventory transactions so that it would default all the affected parts identified by the locate engine for that location by performing a GET operation on the Inventory Count REST service at 704 and the Recall Lot/RecallLotSerial REST services at 705. RF-SMART 408 can be used to perform the physical count, report the count quantity by performing a POST operation at 702 using the same REST services and move them to a quarantine location which prevents further consumption of those parts. While reporting the count, the material transfer happens automatically from the identified location to the quarantine location. There is also a flexibility to use any warehouse management application to inquire the recall data in different locations by leveraging the Inventory Count REST service and Recall Lot/RecallLotSerial REST services which would default the recalled parts information for a given location. This integration in embodiments is built with an extension on the client side of warehouse management applications to report the count quantity against each part or lot or serial in each identified location and pass back the count quantity reported to the ERP Cloud by generating the material transfer transaction to the quarantine location.

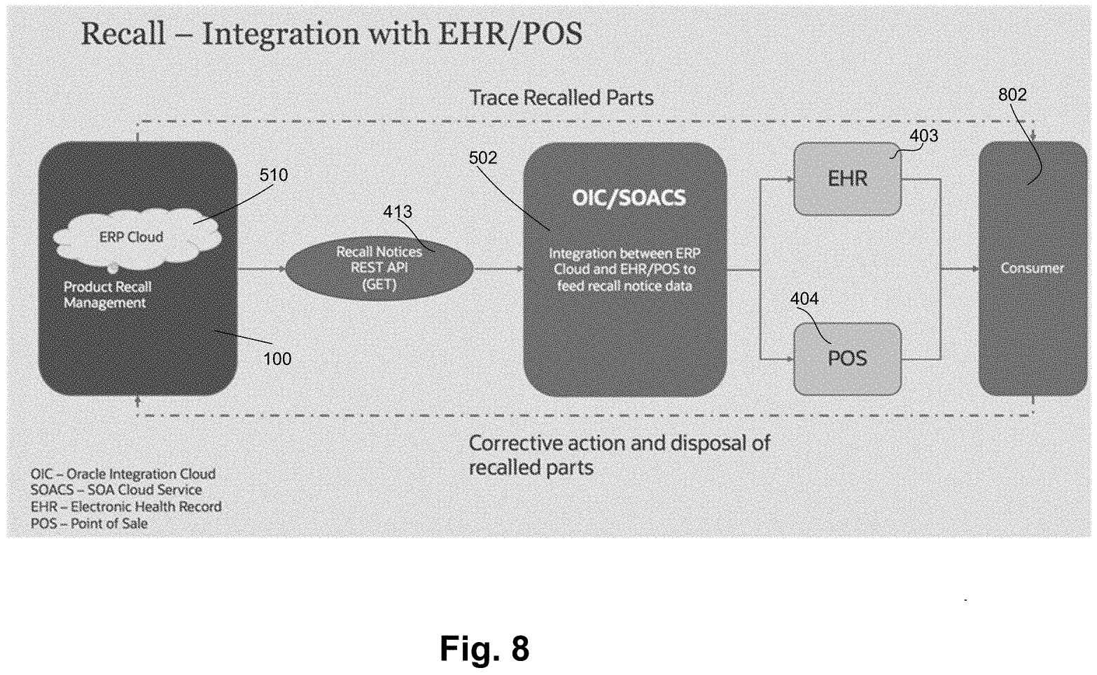

FIG. 8 is a flow diagram illustrating the integration of healthcare product recall system 100 with 3^{rd} party systems to trace recalls and take disposal actions in accordance to other embodiments of the invention. In embodiments, once the materials that are stocked in warehouse locations are issued to an internal pharmacy or issued/implanted (e.g., breast implants) to patients (consumer 802) for consumption, it will be out of track in system 100. Instead, it will get tracked using EHR 403 and POS 404, which are external applications with respect to system 100. Recall Notice REST API 413 is leveraged to integrate ERP 510 with these external applications to track the affected parts that are already issued to patients and take corrective action. With a middleware platform 502 having SOA CS or OIC, Recall Notice REST services 413 can be invoked through a scheduled orchestration as a batch process to get the recall notice data automatically whenever a notice gets imported/published system 100. Recalled manufacturer lots, serials and part numbers captured in system 100 and Oracle ERP cloud 510 will act as the source data that can be mapped to the target which would be an API provided by POS or EHR within the middleware platform so that the recall notice data is fed into these systems to identify to which patient/consumer the recalled part has been issued/implanted.

Embodiments implement multiple specialized REST web services that interface with 3rd parties and users via a REST API request. These REST web services in embodiments are listed below in Table 1:

TABLE 1

| REST Service | Description |
|---|---|
| Recall Notices | The Recall Notices resource manages the information about a recall notice. A recall |
| | notice is created at a business-unit level. The recall notice contains a recall notice |
| | number that gets generated based on the recall document sequence defined in a |
| | configuration parameter. It captures the details of the source that initiated the recall, |
| | the source recall document reference, initiation date, recall reason, recall |
| | classification based on potential risk and the severity of the impact on human life if |
| | consumed, and the replacement method to dispose the recalled parts. The header |
| | identifier is a unique application-generated primary key and is invisible to the user. |
| Recall Contacts | The Recall Contacts resource manages the contact details of person in the recalling |
| | organization. The contact information includes name, position, phone, and email. |
| Recall Lines | The Recall Lines resource manages the information about a recall notice line, which |
| | includes details such as manufacturer part number, part description, recall quantity, |
| | model, brand, internal master item number, item description, and item category. The |
| | line identifier is a unique application-generated primary key and |
| | is invisible to the user. |
| Recall Task | The Recall Task History resource gets the list of tasks and their status that are |
| History | assigned for a product recall at traceability header and at disposition organization |
| | level. This resource can also be used to update the status of the task. |
| Recall Trace | The Recall Trace Status Details resource gets the product traceability information for |
| Status Details | a recalled product after a locate action is performed. A recall notice line can have |
| | multiple trace status detail records, depending on different traceability status in |
| | which the recalled product has been located. The traceability statuses include PAR, |
| | EXPENSE, IN STOCK, and INBOUND. For each traceability status in which the |
| | recalled part in the recall notice line exists or for each warehouse or location or |
| | subinventory where the recalled part is located, there will be a unique trace status |
| | detail record created. If the product traceability status is EXPENSE, the traceability |
| | record can be unique for each requester who is supposed to consume the recalled |
| | parts. The product trace identifier is the unique application generated primary key. |
| | The trace detail status include lot, serial, subinventory code, stock locator, and |
| | transaction details. If the subinventory or stock locator remains same but there are |
| | multiple lots or serials, then there will be as many product traceability detail records |
| | as the number of lots or serials. The product trace line identifier is the unique |
| | application generated primary key. |
| Recall Disposition | The Recall Disposition Details resource gets the disposition details for the recalled |
| Details | parts, which corresponds to each task at the disposition organization level. The |
| | disposition details include disposition method, disposition quantity details, document |
| | number reference with which the disposition action is performed, shipment number |
| | based on which material is returned to vendor, ship to address used, and transaction |
| | date. |
| Inventory Count | The Inventory Count Tasks resource manages the count tasks. Oracle Supply Chain |
| Tasks | for Healthcare Cloud publishes the quantity details for a recalled item to Oracle |
| | Inventory Management Cloud. The count quantity is entered by the counter. This |
| | resource is not currently used. The resource is associated with a feature that requires |
| | opt in. |
| Recall Lot Serials | The Recall Lot Serials resource gets the information about the manufacturer lot and |
| | serial details for each recall notice line primarily used when the recalled item is not |
| | defined with lot and serial control in inventory but having manufacturer lot |
| | and serial captured in the recall notice. |
| Recall Lots | The Recall Lots gets the information about the manufacturer lot details |
| | for each recall notice line primarily used when the recalled item is not |
| | defined with lot and serial control in inventory but having manufacturer lot |
| | and serial captured in the recall notice. |

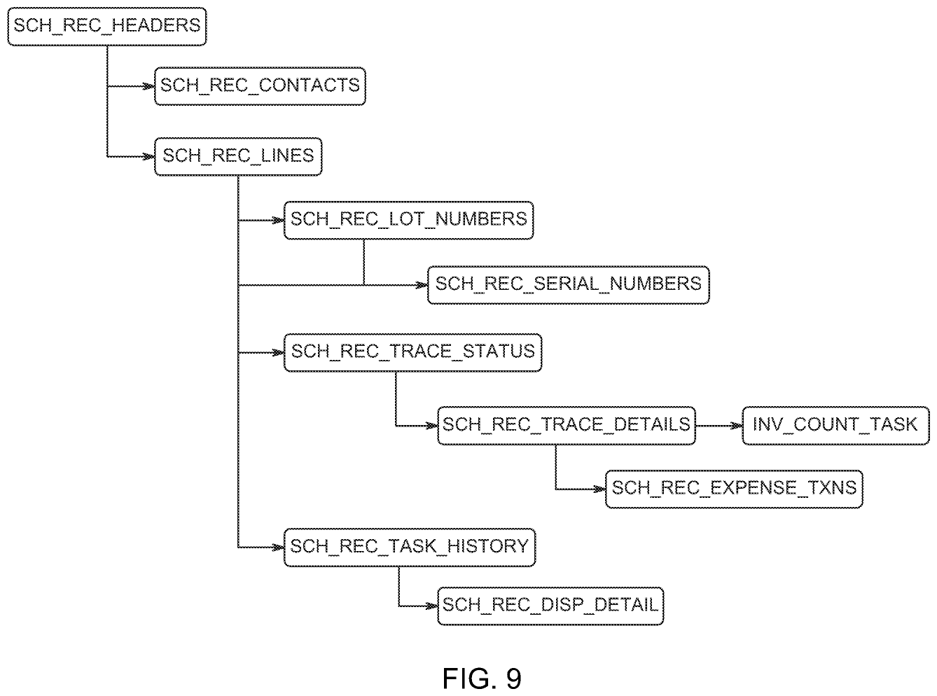

FIG. 9 illustrates specialized data structures implemented in an embodiments of the invention. The specialty data structures can be stored in a database using unique tables and rows and unique attributes. Table 2 below provides details on each of the data structures:

TABLE 2

| Table Name | Description |
|---|---|
| SCH_REC_HEADERS | This table stores the header information of a product recall notice A |
| | recall notice is created at business unit level. Each row contains the |
| | recall notice number that gets generated based on recall document |
| | sequence defined in configuration parameter. It captures the details |
| | of the source who initiated the recall, the source recall document |
| | reference, initiation date and the classification of recall notice |
| | based on nature of the recall reason and the severity of |
| | the impact and the replacement method to dispose the recalled parts. |
| SCH_REC_CONTACTS | This table contains manufacturer contact information |
| | including address and contact details. |
| SCH_REC_LINES | This table stores the line information of a product recall notice which |
| | includes manufacturer part details such as part number, model or |
| | brand. It also stores the internal master item details such as item |
| | number, item description and item category. Each line will have |
| | reference to ITEM_VALIDATION_ORG_ID which is derived |
| | based on the business unit in which recall notice is created. |
| SCH_REC_LOT_NUMBERS | This table stores the recalled manufacturer lot information |
| | that includes lot number, manufacturing date, expiry date for a |
| | recalled item stored against a recall notice line |
| SCH_REC_SERIAL_NUMBERS | This table stores the recalled manufacturer serial information that |
| | includes serial number, lot number, manufacturing date, |
| | expiry date for a recalled item with or without lot stored against |
| | a recall notice line or a lot |
| SCH_REC_TRACE_STATUS | This table stores the product traceability header information for a |
| | recalled product when the Locate action is executed on the recall |
| | notice line. One recall notice line can |
| | have multiple product traceability header records in this |
| | table depending on which all traceability status |
| | the recalled product has and where all they reside. The product |
| | traceability status includes PAR, EXPENSE, |
| | IN STOCK, RECEIVING, IN TRANSIT and SOLD. For |
| | each traceability status in which the recalled part in the recall |
| | notice line exists or for each warehouse or |
| | location or sub inventory where the recalled part resides, there will be |
| | unique product traceability header record which gets created. If the |
| | product traceability status is EXPENSE, the traceability header record |
| | can be unique for each requester who is supposed to consume the |
| | recalled parts. PRODUCT_TRACE_ID is the |
| | unique system generated primary key in this table. |
| SCH_REC_TRACE_DETAILS | This table stores the product traceability detailed information for a |
| | recalled product when the Locate action is executed on the recall |
| | notice line. This product traceability details include lot, serial, sub |
| | inventory code, stock locator and transaction details. One product |
| | traceability header can have multiple |
| | product traceability detail records in this table depending on |
| | which all sub inventories or stock locator the |
| | recalled part resides. If the subinventory or stock |
| | locator remains same but there are multiple lots or serials, |
| | then there will be as many product traceability detail |
| | records as the number of lots or serials. |
| | PRODUCT_TRACE_LINE_ID is the unique system |
| | generated primary key in this table. |
| INV_COUNT_TASK | This table stores the recall trace details against which recall count can |
| | be performed and count quantity can be captured based on which |
| | recall disposition tasks will be progressed for closure. |
| SCH_REC_EXPENSE_TXNS | This table stores the transaction details for recalled items that are |
| | delivered to expense locations. |
| SCH_REC_TASK_HISTORY | This table stores the history of tasks assigned to the product recall |
| | traceability headers or recall notice lines along with the task status |
| | and information on when the task is assigned and when it is |
| | completed. It also keeps a track of whether the notification has been |
| | sent for the assigned task. For each product traceability header or for |
| | each recall notice line, there can be multiple tasks depending on |
| | whether the task is a line level task or traceability level task. Tasks |
| | are pre-seeded with their own unique identifier and stored in a |
| | separate table called “SCH_REC_TASKS_B” and these tasks are |
| | used in this table as per the assignment done at line level and |
| | product traceability header level. ACTION_ID is the unique system |
| | generated primary key in this table. |
| SCH_REC_DISP_DETAIL | This table stores the disposition action details for the recalled parts |
| | that corresponds to each task assigned at the disposition inventory |
| | organization level. This includes disposition method whether it is |
| | Return to Vendor or Scrap, disposition quantity details, document |
| | number based on which disposition action is taken, shipment number |
| | based on which Return to vendor transaction takes place and the |
| | transaction date. |

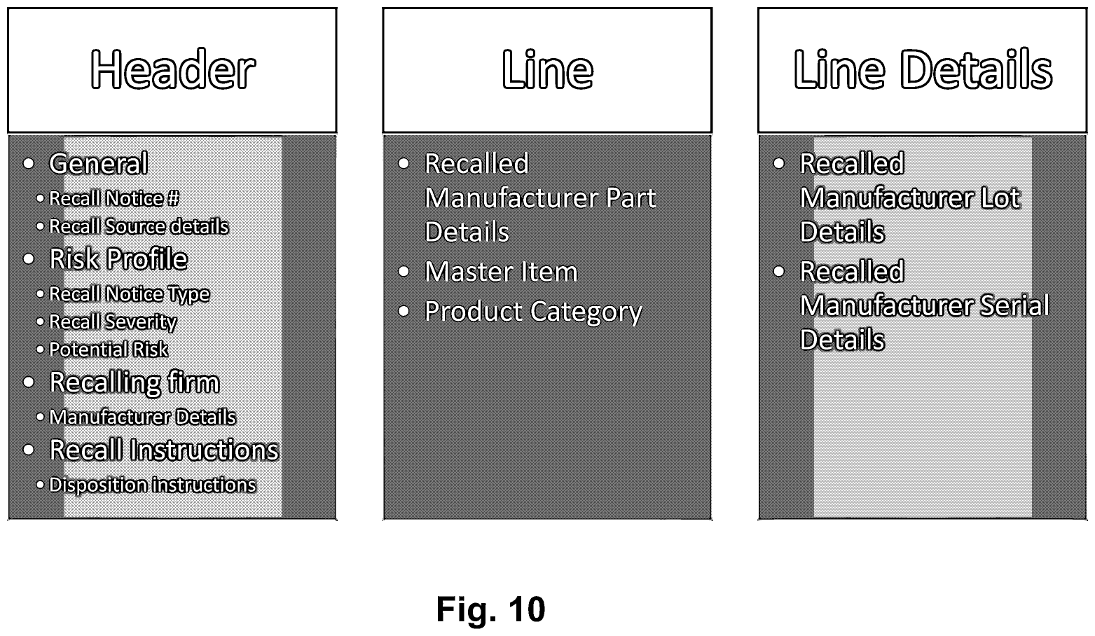

FIG. 10 illustrates the contents of the recall notices in accordance with embodiments. Each recall notice includes a header, a line, and line details, each of which include multiple attributes. The recall notice can be stored in a database as a unique data structure using unique tables and rows and unique attributes.

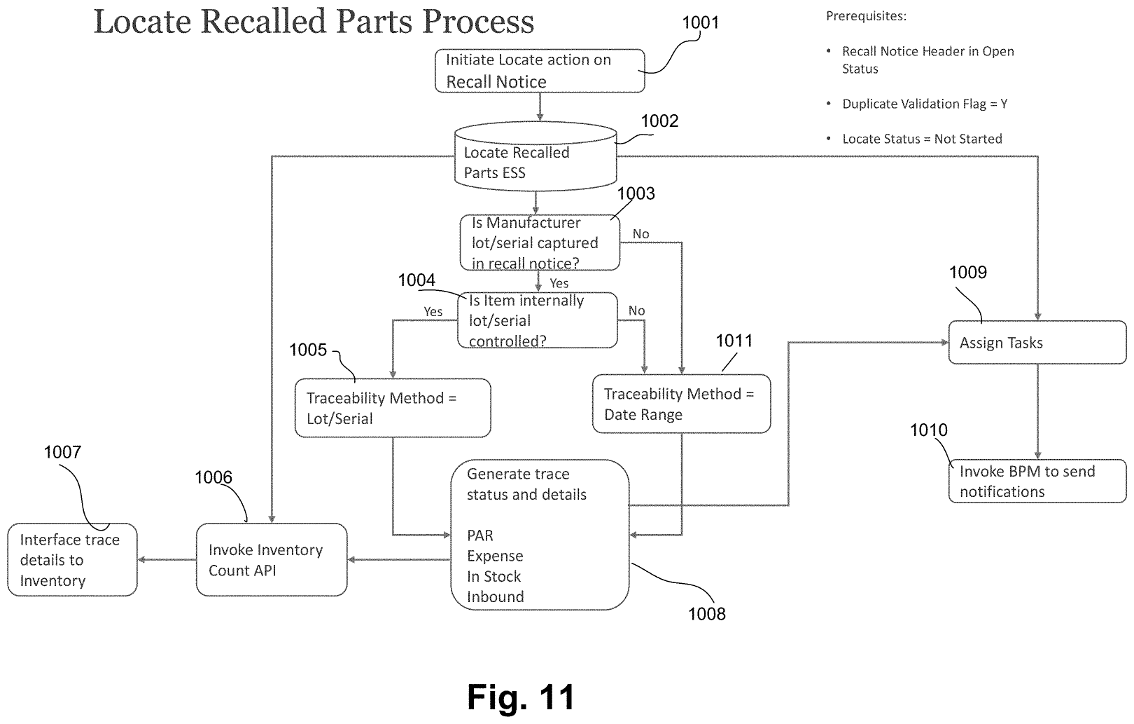

FIG. 11 is a flow diagram of the recall parts process in accordance to embodiments of the invention. The functionality of FIG. 11 implements tracing and tracking of the recalled healthcare products. Specifically, the functionality of FIG. 11 implements locating the recalled parts in the supply chain and providing the details on appropriate product traceability status—PAR, IN STOCK, EXPENSE and INBOUND and in which exact location the parts can be found so that they can be disposed and prevent the users from further consumption.

At 1001, the locate action is initiated on the recall notice that includes the details shown at FIG. 10. Initiating at 1001 triggers at 1002 a locate recalled parts scheduler such as Oracle Enterprise Scheduler (“ESS”) is primarily a Java EE application that provides the user with an ability to schedule any job on-demand or periodic interval. It also facilitates native support to manage the complete life cycle of a job definition: development, distribution, scheduling, and monitoring.

In embodiments, parts that are captured in the recall notice can be either a lot/serial controlled item in an inventory management system such as “Oracle Inventory” or not a lot/serial enabled item regardless of whether the parts are lot/serial controlled in the manufacturer end. If the parts are lot/serial controlled in the inventory management system determined at 1003 and 1004, the process track the parts based on lot # and/or serial #1005. If the parts are not lot/serial controlled in the inventory management system, the process track the parts based on date range captured in the recall notice at 1011. If there is no date range captured in the recall notice, it can be externally provided.

A recall notice can have multiple lines with some parts that are lot/serial controlled in inventory management and some parts that are not. However, the locate action is initiated at the entire recall notice header level and a date range may still be requested. This date range has to be used only for parts that do not have lot/serial number in inventory management.

Table 3 below provides the determination of traceability methods based on lot and serial attributes of the recalled product:

TABLE 3

| Manufacturer Part | Inventory Item | Traceability Method | SKU in trace details |
|---|---|---|---|
| Lot control | Lot control | Traceability based on lot | Insert trace details for each lot |
| Serial control | Serial control | Traceability based on serial | Insert trace details for each lot |
| Lot and Serial | Lot and Serial | Traceability based on serial | Insert trace details for each lot |
| control | control | | and serial |
| Lot control | Lot and Serial | Traceability based on serial | Insert trace details for each lot |
| | control | | and serial |
| Lot control | No Lot or Serial | Traceability by recall track | Insert trace details for item |
| | control | date range | without lot/serial |
| Serial control | No Lot or Serial | Traceability by recall track | Insert trace details for item |
| | control | date range | without lot/serial |
| Lot and Serial | No Lot or Serial | Traceability by recall track | Insert trace details for item |
| control | control | date range | without lot/serial |
| Lot control | Serial control | Traceability by recall track | Insert trace details for each |
| | | date range | serial |
| Serial control | Lot control | Traceability by recall track | Insert trace details for each lot |
| | | date range | |
| No lot or serial | Lot control | Traceability by recall track | Insert trace details for each lot |
| control | | date range | |
| No lot or serial | Serial control | Traceability by recall track | Insert trace details for each |
| control | | date range | serial |
| No lot or serial | Lot and serial | Traceability by recall track | Insert trace details for each lot |
| control | control | date range | and serial |
| No lot or serial | No Lot or Serial | Traceability by recall track | Insert trace details for item |
| control | control | date range | without lot/serial |

Locate by Lot/Serial

With the derived lot/serial number for the recalled part in each inventory org, perform the tracing as follows by passing the parameters—Item ID, Lot #/Serial #,

Organization ID:

1. Verify whether there are any purchase receipts, Subinventory transfer,

Movement Request transfer, Direct Org transfer, In transit receipt, Transfer Order transfer, Transfer Order receipt into non quantity tracked subinventory for the item #, lot # and/or serial #. If this returns records, insert trace headers with the product traceability status as PAR.

2. Verify whether stock exists in quantity tracked subinventory for the item #, lot # and/or serial #. If this returns records, insert trace headers with the product traceability status as IN STOCK.

3. Verify whether there are any Transfer Order issue (Transfer order already received in destination), Movement Request issue, Miscellaneous issue, Account issue, Account Alias issue, User defined Miscellaneous issue for the item #, lot # and/or serial #. If this returns records, insert trace headers with the product traceability status as EXPENSE. All types of Misc. issues (user defined, account alias . . . ) should have the requester populated only based on which the issue transaction would be considered to stamp the traceability status as EXPENSE. Though the item is lot/serial controlled, when there is a direct purchase receipt of this item to expense location, it is not possible to capture the receipt based on lot/serial as the destination is EXPENSE. In such a scenario, the purchase receipts to expense location should be captured for the recalled item based on the date range provided while initiating the LOCATE action.

4. Verify whether there are receipts against in transit shipments, purchase orders or transfer orders which are yet to be delivered to the subinventory for the item #, lot # and/or serial #. If this returns records, insert trace headers with the product traceability status as INBOUND. When there are multiple lots or serials for the same shipment line which have a partial receipt against in transit shipment, it is not possible to identify which lot/serial is in transit and which one is received. In this case also, the traceability status should be stamped as INBOUND. Though the item is lot/serial controlled, when there is a purchase receipt to Receiving dock without any ASN, it is not possible to capture the receipt based on lot/serial. In such a scenario, the purchase receipts to Receiving destination should be captured for the recalled item based on the date range provided while initiating the LOCATE action.

5. Verify whether there are Advanced Shipment Notices (“ASN”) against purchase orders, in transit shipments, transfer orders for the item #, lot # and/or serial #. Also verify whether there are any open purchase orders for the item #. If both the validations return records, insert trace headers with the product traceability status as INBOUND.

Locate by Date Range

For the recalled parts that are not lot/serial controlled in each inventory org, perform the tracing as follows by passing the parameters—Item ID and recall track date range captured at the time of taking the ‘Locate’ action.

The recalled items can be non-lot/serial controlled in Oracle Inventory, but it can be lot/serial enabled from the manufacturer perspective. Therefore, there would be a manufacturer lot/serial number for which recall would have been initiated and captured in recall notice. It does not make sense to say that all the quantities that are received for the recalled item # during the recall date range are recalled parts and need to be disposed. Therefore, estimated recall quantity should be calculated only based on the material that is transacted through defective sub-inventories that will be identified prior to starting the trace of recalled parts.

1. Identification of defective subinventories.

2. Identify all quantity tracked subinventories and stock locators (if exist) across inventory organizations where purchase receipts are done during the recall period and mark them as defective subinventories and locators.

3. Identify all transfers to quantity tracked subinventories and locators (except Recall subinventory) from the defective subinventories and locators from recall track start date till date (Sub transfer with qty <0, Movement request transfer with qty <0, In transit shipment (already received), Direct org transfer with qty <0, TO Shipment (already received), TO Transfer with qty <0). This can be executed recursively for the resultant output till we get 0 records.

Note: Recall Subinventory is the final location where the identified recalled parts will be quarantined and then will get disposed from there. Hence this subinventory is bound to have the recalled parts and should not be considered as defective subinventories.

### Example

Org A has 6 subinventories—S1, S2, S3, S4, S5, S6 and Org B has S7, S8, S9.
Purchase receipts are done during the recall period into OrgA.S1, OrgA.S2 and OrgB.S7. Therefore these 3 subinventories are considered as defective.
Transfers have taken place from OrgA.S1 to OrgA.S3 and OrgA.S1 to OrgB.S8. Therefore these 2 subinventories—OrgA.S3 and OrgB.S8 also became defective
Resultant output OrgA.S3 and OrgB.S8 have also subsequently transferred material to OrgA.S4. Therefore OrgA.S4 also became defective
Resultant output OrgA.S4 has not transferred any material further. Therefore the defective subinventories are OrgA.S1, S2,S3,S4 and OrgB.S7,S8.
Any material for the recalled item # that is lying in OrgA.S5, OrgA.S6, OrgB.S9 are not considered as recall though that item # has a recall notice

4. Insert trace headers with traceability status as IN STOCK when on hand quantity >0 for any of the defective subinventories already identified as per the steps above.

5. Insert trace headers with traceability status as PAR when there are purchase receipts into non quantity tracked subinventories during the recall period.

6. Insert trace headers with traceability status as PAR when there are transfer receipts (Sub transfer with qty >0, Movement request transfer with qty >0, In transit receipt, Direct org transfer with qty >0, TO Receipt, TO Transfer with qty >0) to non quantity tracked subinventories during recall track start date till date. But the transfer should have taken place from the defective subinventory already identified.

7. Insert trace headers with traceability status as EXPENSE if there are receipts against purchase orders of expense destination during the recall period for the recalled item #. In case of description based items, verify whether there are any purchase receipts for the Manufacturer part number. Get the BPA corresponding to recalled MPN and derive the standard po line that refers this BPA line. Verify whether there is any DELIVER transaction to expense location for this standard PO line in the receiving transaction history. Trace records should be populated for MPN as the item number is null for the recall notice having description based items.

8. Insert trace headers with traceability status as EXPENSE if there are issues (TO Issue against transfer order in received status, Misc. Issue, Account Issue, Account Alias Issue, Movement Request Issue, User Defined Misc. Issue) into expense location from the recall track start date till date from the defective subinventory already identified.

9. Insert trace headers with traceability status as INBOUND if there are receipts against purchase orders during the recall period which are yet to be delivered to the subinventory or location.

10. Insert trace headers with traceability status as INBOUND if there are transfer receipts (In transit receipt, TO receipt with inventory destination) from the defective subinventory, during the recall track start date till date which are yet to be delivered to the subinventory or location.

11. Insert trace headers with traceability status as INBOUND if there are ASN against purchase orders during the recall period.

12. Insert trace headers with traceability status as INBOUND if there are open purchase orders for the recalled parts.

13. Insert trace headers with traceability status as INBOUND if there are in transit shipments (interorg shipment, TO shipment and TO Issue) during the recall track start date till date from the defective subinventory already identified.

At 1006, once the trace headers and details are created, the “Locate Recalled Parts” process invokes an inventory count API to populate the trace records in the Inventory count table. Based on the records populated in inventory count table, the Inventory Count user interface will display the records with SKUs against which physical count quantity will be reported by the user. The sequence in which trace records were populated should be followed while populating the Inventory Count table by invoking the Inventory Count API so that the person doing the physical counting need not go around different sub-inventories in a random fashion to perform the counting.

At 1008, as an outcome of the “Locate” action on the recall notice, product traceability statuses are identified with the corresponding source transaction details by the ‘Locate Recalled Parts’ process as explained above. Based on this product traceability status and replacement type specified under Recall Instructions of the recall notice, a sequence of tasks get assigned by the same scheduled process ‘Locate Recalled Parts’ at 1009. These tasks are primarily meant to perform the physical count of recalled parts in the identified locations, quarantine them and subsequently take disposition action such as performing a return to vendor or destroy the parts. There can also be a situation where the action would be to just follow some instruction such as labeling change or device correction which is categorized with the replacement type in the recall notice as “Correction”. This task does not involve any inventory or financial transaction and it is just performing the task as instructed. Depending on the nature of the task, it will be either assigned to the product traceability header or to the disposition organization in which the recalled parts are located. Details of the tasks and the sequence in which they get assigned based on certain conditions are listed in Table 4 below:

TABLE 4

| Product | | | | | Task |
|---|---|---|---|---|---|
| Traceability | Replacement | Task | | | Assignment |
| Status | Type | Sequence | Task Code | Task Name | Level |
| PAR | Return with | 100 | ORA_COUNT | Perform Physical | Traceability |
| | Credit | | | Count and | Header |
| | | | | Quarantine | |
| | | | | Recalled Parts | |
| PAR | Credit Only | 100 | ORA_COUNT | Perform Physical | Traceability |
| | | | | Count and | Header |
| | | | | Quarantine | |
| | | | | Recalled Parts | |
| PAR | Correction | 100 | ORA_COUNT | Perform Physical | Traceability |
| | | | | Count and | Header |
| | | | | Quarantine | |
| | | | | Recalled Parts | |
| PAR | Correction | 110 | ORA_INSTRUCTIONS | Follow Recall | Traceability |
| | | | | Instructions | Header |
| IN STOCK | Return with | 100 | ORA_COUNT | Perform Physical | Traceability |
| | Credit | | | Count and | Header |
| | | | | Quarantine | |
| | | | | Recalled Parts | |
| IN STOCK | Credit Only | 100 | ORA_COUNT | Perform Physical | Traceability |
| | | | | Count and | Header |
| | | | | Quarantine | |
| | | | | Recalled Parts | |
| IN STOCK | Correction | 100 | ORA_COUNT | Perform Physical | Traceability |
| | | | | Count and | Header |
| | | | | Quarantine | |
| | | | | Recalled Parts | |
| IN STOCK | Correction | 110 | ORA_INSTRUCTIONS | Follow Recall | Traceability |
| | | | | Instructions | Header |
| EXPENSE | Return with | 100 | ORA_COUNT | Perform Physical | Traceability |
| | Credit | | | Count and | Header |
| | | | | Quarantine | |
| | | | | Recalled Parts | |
| EXPENSE | Credit Only | 100 | ORA_COUNT | Perform Physical | Traceability |
| | | | | Count and | Header |
| | | | | Quarantine | |
| | | | | Recalled Parts | |
| EXPENSE | Correction | 100 | ORA_COUNT | Perform Physical | Traceability |
| | | | | Count and | Header |
| | | | | Quarantine | |
| | | | | Recalled Parts | |
| EXPENSE | Correction | 110 | ORA_INSTRUCTIONS | Follow Recall | Traceability |
| | | | | Instructions | Header |
| INBOUND | Return with | 100 | ORA_DELIVER | Deliver Recalled | Traceability |
| | Credit | | | Parts to | Header |
| | | | | Quarantine | |
| | | | | Location | |
| INBOUND | Credit Only | 100 | ORA_DELIVER | Deliver Recalled | Traceability |
| | | | | Parts to | Header |
| | | | | Quarantine | |
| | | | | Location | |
| INBOUND | Correction | 100 | ORA_INSTRUCTIONS | Follow Recall | Traceability |
| | | | | Instructions | Header |
| | Return with | 200 | ORA_DISPOSAL | Perform Disposal | Disposition |
| | Credit | | | of Recalled Parts | Inventory |
| | | | | | Organization |
| | Return with | 210 | ORA_DEBIT_MEMO | Raise Debit | Disposition |
| | Credit | | | Memo Against | Inventory |
| | | | | Disposition for | Organization |
| | | | | Recalled Parts | |
| | Credit Only | 200 | ORA_DISPOSAL | Perform Disposal | Disposition |
| | | | | of Recalled Parts | Inventory |
| | | | | | Organization |
| | Credit Only | 210 | ORA_DEBIT_MEMO | Raise Debit | Disposition |
| | | | | Memo Against | Inventory |
| | | | | Disposition for | Organization |
| | | | | Recalled Parts | |

These tasks are seeded and defined in the database level and not exposed to users through the configuration UI. The task sequence for each product traceability status is also seeded and defined in the database level and not exposed to users through the configuration UI. Gaps are provided in the task sequence numbering just to accommodate more seeded tasks in between the existing sequence if there is a business need in future. Based on the replacement type in the recall notice, the appropriate task for a product traceability status will be picked from the task assignment seed data.

FIG. 12 illustrates functionality of the work area that provides a user with guided actions and appropriate actions to be taken on a recall notice based on its status and helps the user navigate to different user interfaces to manage the notices in accordance to embodiments.

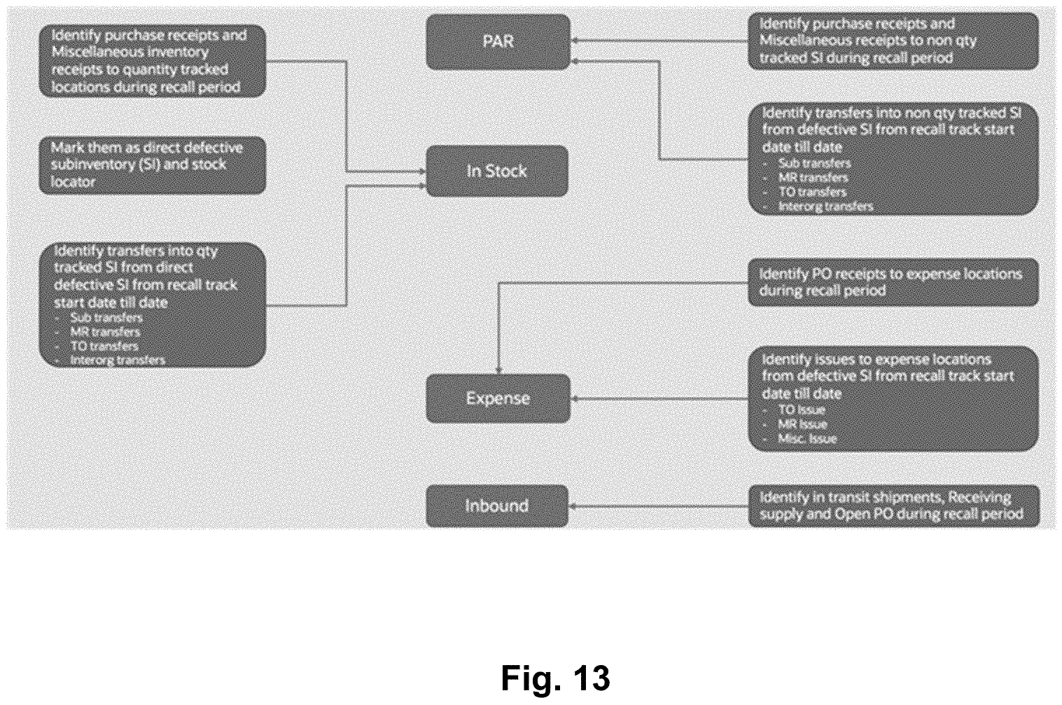

FIG. 13 illustrates functionality of the locate recalled parts engine for locating recalled parts that are not tracked using a lot or serial number in accordance to embodiments.

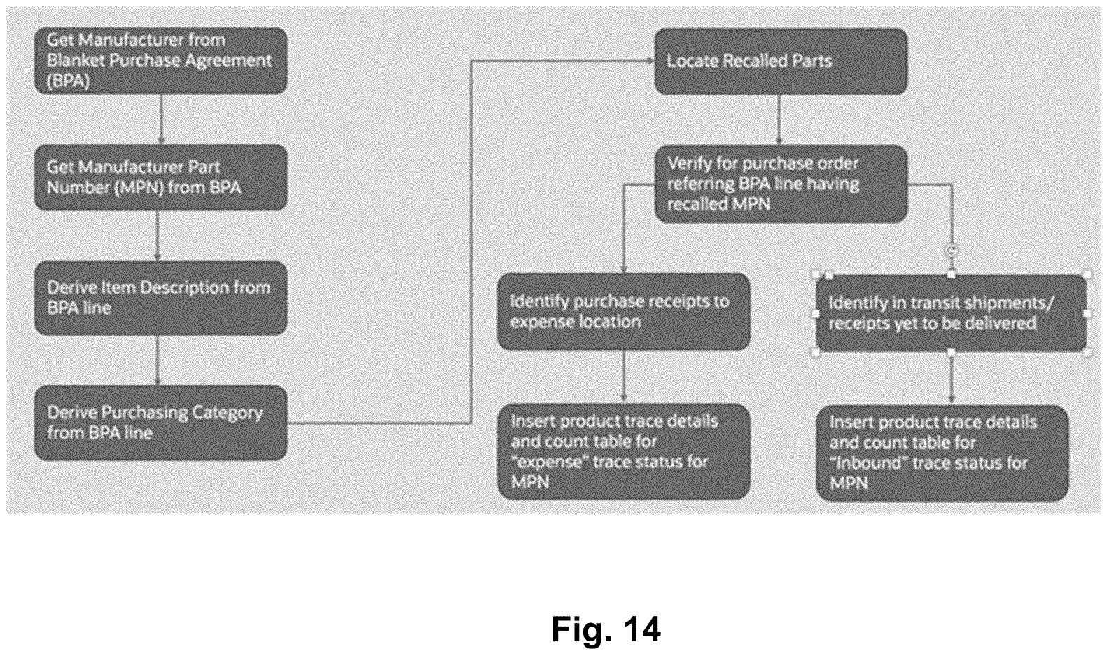

FIG. 14 illustrates functionality of the locate recalled parts engine for locating recalled parts that do not have an internal item number in accordance to embodiments.

As disclosed, healthcare product recall system 100 provides for an integration of an ERP system with a regulatory authority or any third-party applications in real time using REST APIs and file-based data imports to capture recall notices on-time rather than receiving the notifications manually from multiple sources which introduces delays. Embodiments include extensive traceability of recalled parts across different locations in the enterprise. The locate engine not only tracks the recalled parts in stock but also traces the parts lying in non-quantity tracked locations, thereby ensuring consumer safety. Embodiments automatically assign the appropriate recall tasks, segregate them based on departments and notify resources to dispose the affected parts. This avoids the need to locate the affected parts in the perpetual inventory locations and non-quantity tracked locations and then quarantine them for disposal.

Embodiments include automated notifications driven by flexible business rules that provide alerts to the stakeholders in various departments where the recalled parts are located so that they can carry out the assigned tasks required for disposal. Embodiments provide integration with devices such as RF-SMART, enabling a recall count using hand-held device. Any third-party logistics execution systems can leverage the recall count REST services to report the count quantity in identified locations and to quarantine them. Embodiments provide a magnified view on the traceability details and the tasks assigned, enabling a user to monitor the automatic tasks progression in different departments and locations. This in turn provides more visibility and control over what actions are taken on the recalled goods. There is no need of manual follow up with the warehouse personnel to perform the disposition tasks.

REST services provide by embodiments integrate an ERP system with EHR or POS to trace the patients affected by the recall and take corrective action rather than manually checking the implant logs or EHR.

Embodiments provide a magnified view on the traceability details and the tasks assigned, enabling the monitoring if the automatic tasks progression in different departments and locations. A user can review the trace details for a specific item or specific location with different traceability statuses. The user can also track the progress for a specific task across different locations. For example, a user can see in which all departments, the count task is in progress at a given point of time and in which departments the task is already completed. A predefined set of tasks get assigned automatically based on the traceability status and replacement type and those tasks automatically get progressed and closed based on certain validations. For example, a Count task will get closed only when the recall count quantity is reported in all identified locations. A Disposal task will get closed only when the disposition quantity matches with the count quantity for a given facility. There is no need of manual intervention to close the recall notice as it would automatically get closed when all the predefined set of tasks get closed based on the above-mentioned validations.

Several embodiments are specifically illustrated and/or described herein. However, it will be appreciated that modifications and variations of the disclosed embodiments are covered by the above teachings and within the purview of the appended claims without departing from the spirit and intended scope of the invention.

What is claimed is:

1. A method of managing the recall of a healthcare product comprising a plurality of healthcare parts, the method comprising:
capturing a recall notice for the healthcare product;
in response to receiving a locate action from a user, initiating by a locate engine a background process for locating each of the healthcare parts;
the locate engine background process identifying a product traceability status for each of the healthcare parts, the traceability status comprising one of: Periodic Automated Replenishment (PAR), in stock, expense, or inbound, the locate engine locating the healthcare parts based on the traceability status;
based on the product traceability status, generating a sequence of tasks based on the traceability status, the tasks implementing a recall action; and
monitoring the completion of the tasks to verify that the recall notice can be closed.

2. The method of claim 1, wherein the recall notice is captured via a Representational State Transfer (REST) Application Programming Interface (API).

3. The method of claim 1, wherein the recall notice is captured via a File Based Data Import (FBDI).

4. The method of claim 1, the sequence of tasks comprising a count task comprising performing a recall count in the identified locations of the healthcare parts and moving the counted parts to a quarantine location, a deliver task comprising delivering the recalled parts to quarantine location, a disposal task comprising performing a return to vendor or scrap disposal, a debit memo task comprising creating a debit note against the disposal performed for the recalled parts and an instructions task comprising carrying out recall instructions.

5. The method of claim 1, wherein the traceability status comprises a data structure comprising a header and child, the header comprising product traceability status, warehouse, location, sub-inventory (optionally based on configuration) and total transaction quantity and the child comprising sub-inventory, stock locator and transaction details, the header and child comprising attributes stored in a specialized database table.

6. The method of claim 1, wherein the healthcare product comprises lot and serial attributes, further comprising the locate engine background process determining a traceability method based on whether the lot and serial attributes are captured in the recall notice.

7. The method of claim 6, wherein the determined traceability method comprises tracing by lot and serial numbers or by a date range captured in the recall notice.

8. The method of claim 5, the specialized database table comprising an inventory count table, the method comprising populating trace records in the inventory count table via an inventory count API.

9. A healthcare product recall management system for managing the recall of a healthcare product comprising a plurality of healthcare parts, the system comprising:
an interface for capturing a recall notice for the healthcare product;
one or more processors executing instructions and adapted to:
in response to receiving a locate action from a user, initiating by a locate engine a background process for locating each of the healthcare parts;
the locate engine background process identifying a product traceability status for each of the healthcare parts, the traceability status comprising one of: Periodic Automated Replenishment (PAR), in stock, expense, or inbound, the locate engine locating the healthcare parts based on the traceability status;
based on the product traceability, generate a sequence of tasks based on the traceability status, the tasks implementing a recall action; and
monitor the completion of the tasks to verify that the recall notice can be closed.

10. The system of claim 9, wherein the recall notice is captured via a Representational State Transfer (REST) Application Programming Interface (API) interface.

11. The system of claim 9, wherein the recall notice is captured via a File Based Data Import (FBDI) interface.

12. The system of claim 9, the sequence of tasks comprising a count task comprising performing a recall count in the identified locations of the healthcare parts and moving the counted parts to a quarantine location, a deliver task comprising delivering the recalled parts to quarantine location, a disposal task comprising performing a return to vendor or scrap disposal, a debit memo task comprising creating a debit note against the disposal performed for the recalled parts and an instructions task comprising carrying out recall instructions.

13. The system of claim 9, further comprising a specialized database table of a database;
wherein the traceability status comprises a data structure comprising a header and child, the header comprising product traceability status, warehouse, location, sub-inventory (optionally based on configuration) and total transaction quantity and the child comprising sub-inventory, stock locator and transaction details, the header and child comprising attributes stored in the specialized database table.

14. The system of claim 9, wherein the healthcare product comprises lot and serial attributes, further comprising the locate engine background process determining a traceability method based on whether the lot and serial attributes are captured in the recall notice.

15. The system of claim 14, wherein the determined traceability method comprises tracing by lot and serial numbers or by a date range captured in the recall notice.

16. The system of claim 13, the specialized database table comprising an inventory count table, further comprising populating trace records in the inventory count table via an inventory count API.

17. A computer-readable medium storing instructions which, when executed by at least one of a plurality of processors, cause the processors to manage the recall of a healthcare product comprising a plurality of healthcare parts, the managing comprising:
capturing a recall notice for the healthcare product;
in response to receiving a locate action from a user, initiating by a locate engine a background process for locating each of the healthcare parts;
the locate engine background process identifying a product traceability status for each of the healthcare parts, the traceability status comprising one of: Periodic Automated Replenishment (PAR), in stock, expense, or inbound, the locate engine locating the healthcare parts based on the traceability status;
based on the product traceability status, generating a sequence of tasks based on the traceability status, the tasks implementing a recall action; and
monitoring the completion of the tasks to verify that the recall notice can be closed.

18. The computer-readable medium of claim 17, wherein the recall notice is captured via a Representational State Transfer (REST) Application Programming interface (API).

19. The computer-readable medium of claim 17, wherein the recall notice is captured via a File Based Data Import (FBDI).

20. The computer-readable medium of claim 17, the sequence of tasks comprising a count task comprising performing a recall count in the identified locations of the healthcare parts and moving the counted parts to a quarantine location, a deliver task comprising delivering the recalled parts to quarantine location, a disposal task comprising performing a return to vendor or scrap disposal, a debit memo task comprising creating a debit note against the disposal performed for the recalled parts and an instructions task comprising carrying out recall instructions.
